(19) United States
(12) Patent Application Publication
(10) Pub. No.: US 2025/0152291 A1
(43) Pub. Date: May 15, 2025

(54) HAIR PRESERVING SURGICAL DEVICE

(71) Applicant: The Trustees of Indiana University, Bloomington, IN (US)

(72) Inventors: Jignesh Tailor, Carmel, IN (US); Steven Wakeman, Tampa, FL (US)

(21) Appl. No.: 18/941,218

(22) Filed: Nov. 8, 2024

Related U.S. Application Data
(60) Provisional application No. 63/597,859, filed on Nov. 10, 2023.

(51) Int. Cl. A61B 90/00; A45D 24/02; A61B 17/17

(52) U.S. Cl. CPC A61B 90/00; A61B 17/171; A61B 17/1739; A45D 24/02

(57) ABSTRACT

A hair preservation surgical device configured to form and at least partially maintain a part or separation among a plurality of hair strands of a patient. A plurality of hair engagement bodies of the device can engage and pull a portion of the plurality of hair strands via displacement of the surgical device, including displacement resulting from deformation of the device, to at least assist in retaining the engaged hair strands away from the part. The surgical device can further be displaced such that an access aperture of the device is positioned over the part. The access aperture can extend through the surgical device and provide a location for performances of surgical procedures at, and through, the location of the part. Attachment mechanisms can be used to engage one or more anchors of the device to selectively secure the device to the patient, including to the scalp of the patient.

### TECHNICAL FIELD

The present disclosure generally relates to a surgical device that facilitates performance of surgical procedures at least at an incision site in a patent's scalp while also preserving the patient's hair.

### BACKGROUND

At least certain types of surgical procedures involve gaining access to, or through, a patient's skull. For example, stereotactic epilepsy surgery typically involves drilling a plurality of holes into a patient's skull, with each hole receiving insertion of an electrode. Traditionally, such surgeries have involved shaving at least some, if not all, of the hair from the patient's head, or select areas of the patient's head. Such shaving of hair can be intended to prevent the hair from interfering, including becoming entangled, with a medical tool, including but not limited to, a cranial drill or associated cranial drill bit that is used to drill holes into the patient's skull.

In addition to at least potentially dealing with an underlying medical issue that is the basis for undergoing such surgical procedures, the removal of hair from the heads of at least certain patients can also be detrimental to those patients' mental well-being, including self-esteem. Additionally, attempts to conceal such hair removal, such as, for example, via the wearing of a wig and/or hats, can further exacerbate the issue, as well as involve the patient incurring additional expenses.

Alternatively, in some instances, a surgeon may physically manipulate the hair of the patient while another surgeon performs the medical procedure. However, such inclusion of an additional surgeon for controlling the patient's hair can increase the cost of the medical procedure, as well as not fully utilize the skill of that surgeon. Further, such an approach is dependent on the surgeon maintaining firm control of the hair in each area in which a drill or incision is placed, which potentially can become problematic with respect to the repeatability and the duration of such procedures.

### SUMMARY

The present disclosure may comprise one or more of the following features and combinations thereof.

In one embodiment of the present disclosure, an apparatus is provided for forming a part along, and controlling a position of, at least a portion of a plurality of strands of hair of a patient. The apparatus can include a handle portion configured to be grasped by at least one of a digit or a palm of a user, and a body portion coupled to the handle portion. The body portion can comprise a plurality of hair retention bodies that can be positioned and configured to engage and retain a position of the plurality of strands of hair. The apparatus can further include an access aperture that extends through at least one of the body portion and the handle portion and is positioned to extend over the part. Further, the apparatus can include at least one anchor configured to be at least partially straddled by an attachment mechanism that secures the apparatus to a head of the patient.

In another embodiment of the present disclosure, a method is provided for engaging a plurality of hair strands of a patient with at least one of a plurality of hair engagement bodies of a hair preservation surgical device. The plurality of engaged hair strands can be pulled via displacement of the hair preservation surgical device to form a part between the plurality of engaged hair strands and other hair strands of the patient. Further, an access aperture of the hair preservation surgical device can be positioned above the part, and one or more attachment mechanisms can engage one or more anchors of the hair preservation surgical device to securely couple the hair preservation surgical device to the patient.

These and other features of the present disclosure will become more apparent from the following description of the illustrative embodiments.

### BRIEF DESCRIPTION OF THE FIGURES

The invention described herein is illustrated by way of example and not by way of limitation in the accompanying figures. For simplicity and clarity of illustration, elements illustrated in the figures are not necessarily drawn to scale. For example, the dimensions of some elements may be exaggerated relative to other elements for clarity. Further, where considered appropriate, reference labels have been repeated among the figures to indicate corresponding or analogous elements.

FIGS. 1 and 2 illustrate top and top side perspective views, respectively, of an exemplary hair preservation surgical device according to an illustrated embodiment of the subject disclosure.

FIG. 3 illustrates a first side view of the hair preservation surgical device shown in FIG. 1.

FIG. 4 illustrates a portion of the hair engagement bodies of the hair preservation surgical device, as taken from box A in FIG. 2.

FIG. 5 illustrates an access aperture of the hair preservation surgical device, as taken from box B in FIG. 2.

FIG. 6 illustrates a first anchor assembly of the hair preservation surgical device, as taken from box C in FIG. 2.

FIG. 7 illustrates a side view of an exemplary hair preservation surgical device being at least partially deformed, deflected, or bent along a central longitudinal axis of the hair preservation surgical device during use of the surgical device.

FIG. 8 illustrates a top view of the hair preservation surgical device shown in FIG. 1 with a plurality of attachment mechanisms positioned about at least some of the anchors of the surgical device and secured to a scalp in a manner that assists in maintaining a part in the patient's hair.

FIG. 9 illustrates a top view of another exemplary hair preservation surgical device having a pair of anchors projecting from opposing portions of the hair preservation surgical device.

FIG. 10 illustrates a top view of an alternative anchor for an embodiment of the hair preservation surgical device.

### DETAILED DESCRIPTION OF THE ILLUSTRATIVE EMBODIMENTS

The following Detailed Description refers to the accompanying drawings that illustrate exemplary embodiments. Other embodiments are possible, and modifications can be made to the embodiments within the spirit and scope of this description. Those skilled in the art with access to the teachings provided herein will recognize additional modifications, applications, and embodiments within the scope thereof and additional fields in which embodiments would be of significant utility. Therefore, the Detailed Description is not meant to limit the embodiments described below.

In the Detailed Description herein, references to “one embodiment”, an “embodiment”, and “example embodiment”, etc., indicate that the embodiment described may include a particular feature, structure, or characteristic, by every embodiment may not necessarily include the particular feature, structure, or characteristic. Moreover, such phrases are not necessarily referring to the same embodiment. Further, when a particular feature, structure, or characteristic may be described in connection with an embodiment, it may be submitted that it may be within the knowledge of one skilled in art to affect such feature, structure, or characteristic in connection with other embodiments whether or not explicitly described.

Embodiments of the subject application generally relate to a hand held hair preservation surgical device that is configured to be manipulated and selectively removably coupled to at least the scalp of a patient in a manner that maintains a part or separation of the hair of the patient at an incision site in the patient's scalp. The hair preservation surgical device further controls the hair in a manner that prevents the hair from interfering with a surgical procedure at, and through, the incision site, including preventing hair from becoming entangled with a cranial drill bit. Accordingly, the hair preservation surgical device includes an access aperture or gap that can accommodate passage of one or more, if not all, of the following: passage of at least a cutting end or blade of a scalpel or other cutting tool that cuts an incision in the patient at the incision site; a cranial drill bit that enters the incision and forms a bore or hole in at least the skull of the patient; an electrode/bolt that is inserted into the drilled bore; and, a surgical tool or device to close the incision site, among other surgical implements, tools, or devices, as well as various combinations thereof.

According to the illustrated embodiments, the hair preservation surgical device also includes one or more anchors, which may be housed in, or outside of, recesses or openings, as well as combinations thereof, of the hair preservation surgical device. The anchors can be configured for engagement with, or by, attachment means that can securely couple, as well as selectively retain, the hair preservation surgical device to at least the patient's scalp, among other portions of the body of the patient.

The hair preservation surgical device can also be a single use, or disposable device. Further, the hair preservation surgical device may, or may not, be classified a Food and Drug Administration (FDA) Class 1 device. The hair preservation surgical device is suitable for use in a variety of surgical procedures, including, not limited to, use in at least attempting to maintain an incision site generally clear of, or from interference by, hair during procedures relating to electrode placement, and, later, electrode removal, for stereotactic epilepsy surgery.

Such above features of the hair preservation surgical device, among other features, can potentially decrease surgical procedure time. Additionally, when securely coupled to the patient via use of one or more of the attachment mechanisms, the hair preservation surgical device can maintain a part or separation of the hair of a patient at the incision site in a manner that may eliminate the need of a second surgeon to control the patient's hair, which may simplify the surgical procedure. Moreover, the hair preservation surgical device can facilitate a single surgeon being able to both maintain the incision site(s) so as to be generally clear of interference from hair, as well as handle placement of stereotactic electroencephalogram electrodes, among other electrodes or medical devise, on hair-bearing scalps without shaving or clipping the hair.

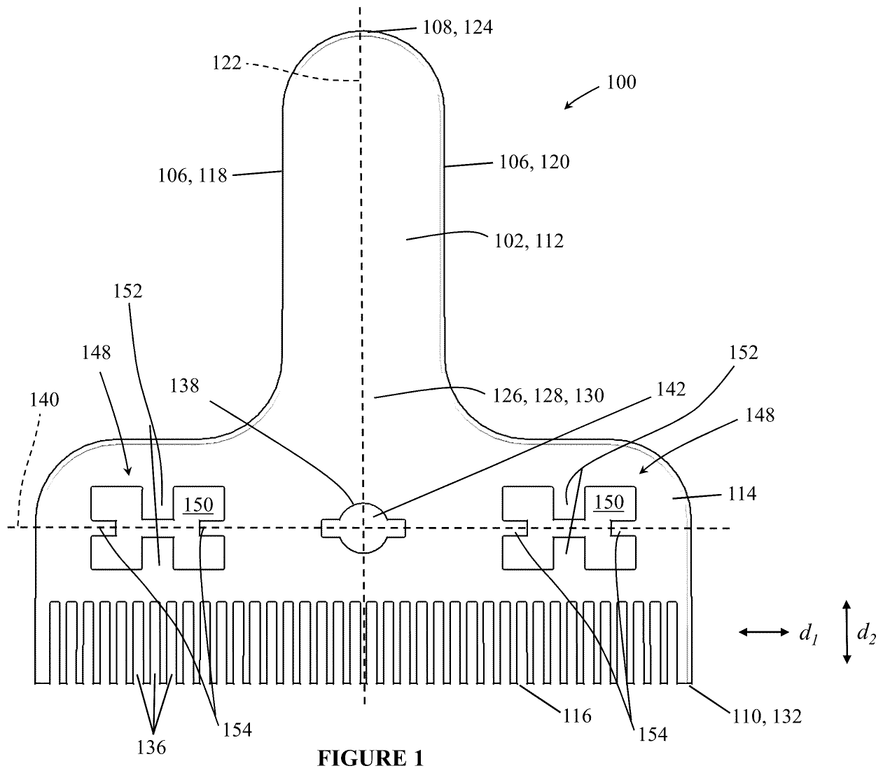

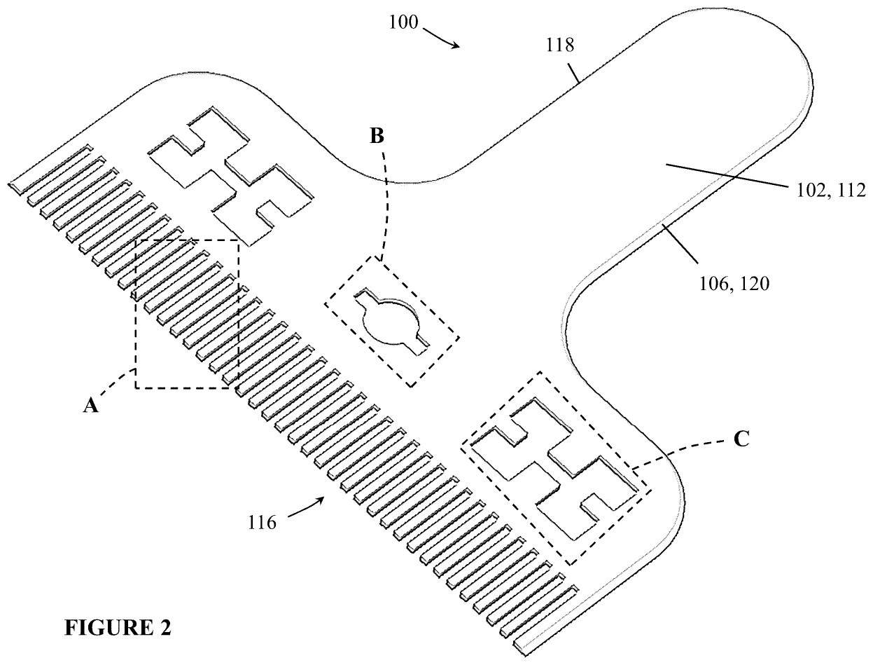

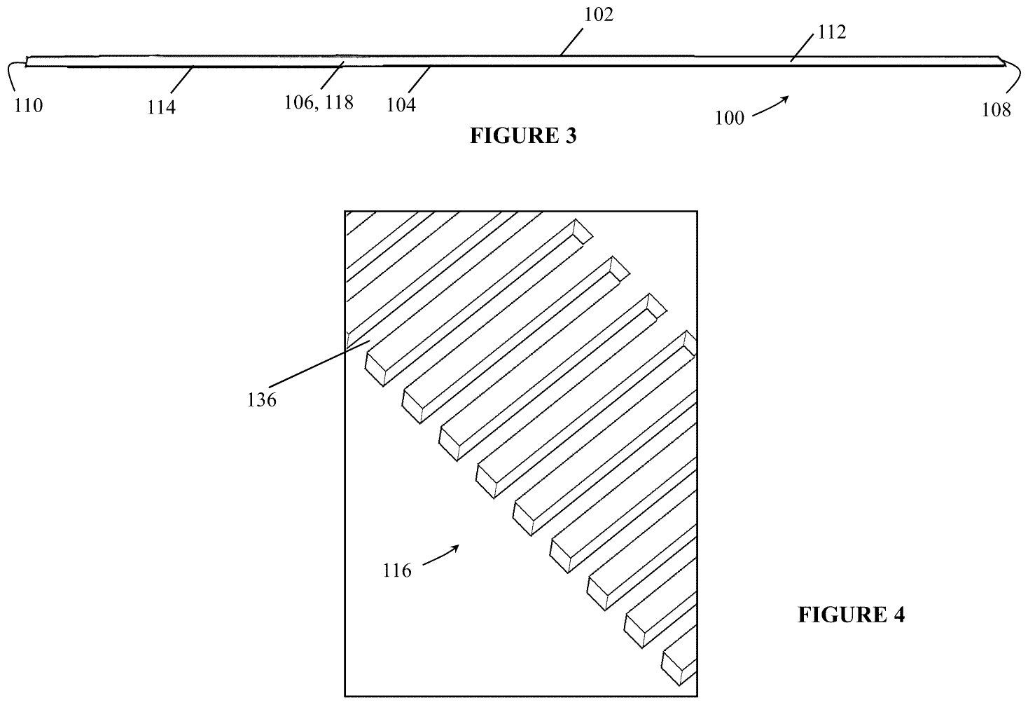

FIGS. 1 and 2 illustrate top and top side perspective views, respectively, of an exemplary hair preservation surgical device 100 according to an illustrated embodiment of the subject disclosure. According to certain embodiments, the bottom and bottom side perspective views of the hair preservation surgical device 100 can be identical to, or a mirror images of, the top views shown in FIGS. 1 and 2, respectively. Similarly, while FIG. 3 illustrates a first, such as right, side view of the hair preservation surgical device 100 shown in FIG. 1, a second, or left, side view of the illustrated the hair preservation surgical device 100 can be identical to, or a mirror image of, the first side view shown in FIG. 3.

Referencing FIGS. 1, 2, and 3, the hair preservation surgical device 100 can include opposing top and bottom walls 102, 104 that can be separated, and joined by, a sidewall 106. As seen at least FIG. 3, according to certain embodiments, both the top wall 102 and the bottom wall 104 can generally be flat planar surfaces that extend from a first end 108 to an opposing second end 110 of the hair preservation surgical device 100. Additionally, according to certain embodiments, the top wall 102 can be generally parallel to the bottom wall 104. The sidewall 106 can have a variety of different configurations, including, be a generally contoured surface that can provide a transition between the top and bottom walls 102, 104.

The hair preservation surgical device 100 can further comprise a handle portion 112 and a body portion 114 having a plurality of hair engagement bodies 116. The handle portion 112 is configured to be grasped by a hand of a user, including, for example, by a plurality of digits or palm of the user, as well as combinations thereof. Thus, the handle portion 112 can be configured such that the user can firmly grasp the handle portion 112 so as to manipulate the position of the hair preservation surgical device 100. Thus, as seen by at least FIG. 1, according to certain embodiments, the handle portion 112 can have a width between a first portion 118 of the sidewall 106 and an opposing second portion 120 of the sidewall 106 (in a direction generally indicated by “d₁” in FIG. 1) that is narrower than a corresponding width of the body portion 114. Additionally, the handle portion 112 can have a length in a direction that is generally parallel to a central longitudinal axis 122 of the hair preservation surgical device 100 that is longer (in a direction generally indicated by “d₂” in FIG. 1) than a corresponding length of the body portion 114. Additionally, according to certain embodiments, the hair preservation surgical device 100 can be generally symmetrical about the central longitudinal axis 122.

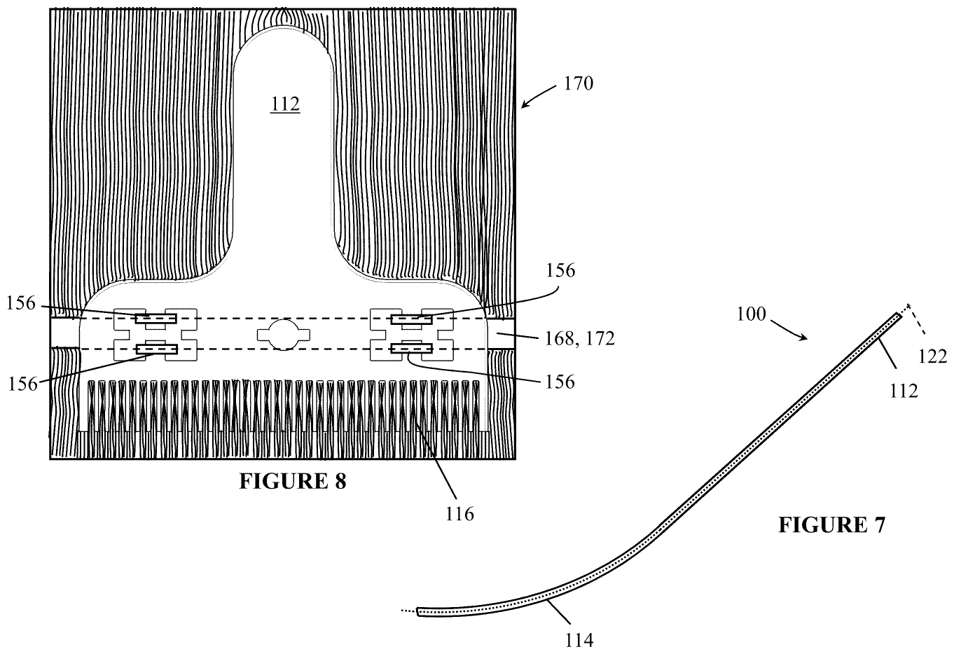

While FIG. 3 illustrates the handle portion 112 and body portion 114 having the same general thickness between the opposing top and bottom walls 102, 104, according to other embodiments, the width of the handle portion 112 can be different than the body portion 114. For example, according to certain embodiments, the handle portion 112 may have a larger thickness between the opposing top and bottom walls 102, 104 then the body portion 114 so as to assist with the ease at which the user can securely, and comfortably, grasp and manipulate the hair preservation surgical device 100. Additionally, according to certain embodiments, the thickness of the hair preservation surgical device 100, as well as the material used for the construction of the hair preservation surgical device 100, can be sized to accommodate a degree of bending, deformation, or deflection of the hair preservation surgical device 100, as illustrated, for example, in FIG. 7, and as discussed below. Thus, according to certain embodiments, the hair preservation surgical device 100 can be constructed from materials that it can accommodate bending, deformation or deflection during normal use of the hair preservation surgical device 100 with minimal risk of cracking or other breakage of the hair preservation surgical device 100.

The handle portion 112 can extend in a direction generally parallel to the central longitudinal axis 122 from a first end 124 of the handle portion 112 to an opposing second end 126 of the handle portion 112. The second end 126 of the handle portion 112 can generally be adjacent to a first end of the body portion 114. According to certain embodiments, the hair preservation surgical device 100 can include a transition region 128 that may be a separate area or a portion of either or both the handle portion 112 and the body portion 114 that provides a transition between the handle portion 112 and the body portion 114, and moreover, provides a transition between at least the differences in widths of the handle portion 112 and the body portion 114. For example, according to embodiments shown in FIGS. 1 and 2, the transition region 128 can provide a gradual increase in the width of the hair preservation surgical device 100 between the handle portion 112 and the body portion 114. Such a transition region 128 can include generally curved or contoured areas between the handle portion 112 and the body portion 114 that can minimize the potential for development of cracks between the handle portion 112 and the body portion 114 that could otherwise develop during the bending, deflection, or deformation of the hair preservation surgical device 100.

The body portion 114 can extend from a first end 130 of the body portion 114 that is generally adjacent to either, or both, the handle portion 112 and the transition region 128, and a second end 132 that is located at and end portion 134 of the sidewall 106. Additionally, according to the illustrated embodiment, each of the hair engagement bodies 116 can inwardly extend from the second end 132 of the body portion 114 in a direction that is generally parallel to the central longitudinal axis 122. Further, according to certain embodiments, the hair engagement bodies can be a plurality of teeth, with each tooth being separated from an adjacent tooth by a space 136, the spaces 136 extending through the end portion 134 of the sidewall 106. Thus, the hair engagement bodies 116 can be configured to generally guide hair, including individual hairs or groups of hair (collectively referred to herein a hair strands) into the spaces 136 between the hair engagement bodies 116 as the hair preservation surgical device 100 is displaced relative to the hair strands of the patient. Additionally, the teeth, and the spaces 136 therebetween, can be sized so as to at least assist in retaining the hair strands in engagement with the hair preservation surgical device 100 such that, as the hair preservation surgical device 100 remains at a generally static position relative to the scalp of the patient, the engaged hair strands are generally retained in a generally taut condition at a position between the spaces 136 and the hair engagement bodies 116.

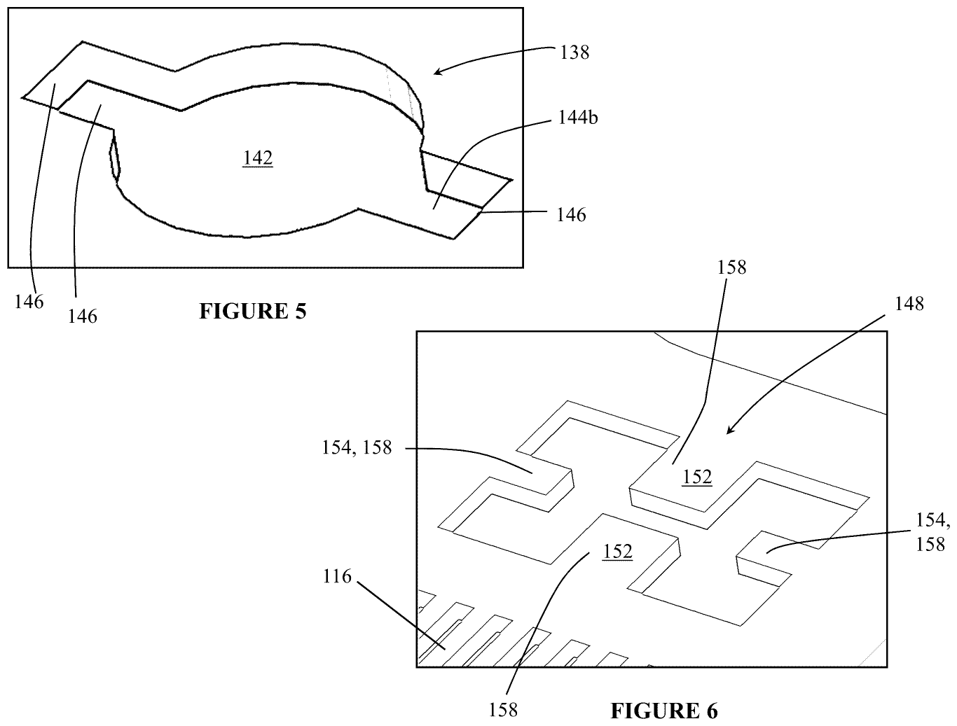

As seen at least in FIGS. 1 and 5, the hair preservation surgical device 100 can include an access aperture 138 through which one or more surgical procedures can be performed. In the illustrated embodiment, the access aperture 138 extends through the body portion 114 of the hair preservation surgical device 100, and more specifically, through both the top and bottom walls 102, 104 of the hair preservation surgical device 100. Alternatively, the access aperture 138 can be positioned to extend through the top and bottom walls 102, 104 at the handle portion 112, or at a location that overlaps the handle and body portions 112, 114, as well as any portion of the hair preservation surgical device 100 therebetween. While the access aperture 138 can be located at a variety of different locations, according to the illustrated embodiment, the access aperture 138 is positioned about a midline 140 of the body portion 114, and can be centrally located and a location at which the central longitudinal axis 122 intersects the midline 140. However, the access aperture 138 can also be positioned at a variety of other locations about the hair preservation surgical device 100.

Referencing FIG. 5, the access aperture 138 can include a generally circular orifice 142 that can have a size to accommodate passage of a cranial drill bit. Thus, for example, the orifice 142 of the access aperture 138 can have a diameter that is slightly larger than a diameter of a corresponding cranial drill bit. The access aperture 138 can further include a pair of slots 144a, 144b that can extend in opposing directions from each other. Thus, for example, according to the illustrated embodiment, each slot 144a, 144b extends in opposing directions from the orifice 142 along the midline 140. However, the slots 144a, 144b can be at a variety of other angular orientations relative to the midline 140 or central longitudinal axis 122. The slots 144a, 144b can be sized to receive movement of a cutting end of a cutting tool, such as, for example, a cutting or blade end of a scalpel. Thus, the slots 144a, 144b can have a linear distance between an end 146 of the first slot 144a, across the orifice 142, and the end 146 of an opposing second slot 144b that can generally correspond to a size of an incision for an incision site that the cutting tool is to cut into at least the patient's scalp. Moreover, the distance between the ends 146 of the first and second slots 144a, 144b can generally guide, if not limit, the size of the incision site that can be cut by the cutting tool.

The hair preservation surgical device 100 can further include at least one anchor that can be engaged by an attachment mechanism, such as, for example, a staple or suture, among other devices that can secure the hair preservation surgical device 100 to the head, or scalp, of the patient. In the embodiment shown in at least FIG. 1, the hair preservation surgical device 100 includes a pair of anchor assemblies 148, namely a first anchor assembly 148 and a second anchor assembly 148 positioned on opposing sides of the central longitudinal axis 122. Further, according to the illustrated embodiment, each anchor assembly 148 can be generally centrally located relative to the midline 140 of the body portion 114. However, such anchor assemblies 148 can also be positioned in a variety of other locations, or combinations of locations, about the body portion 114 or other portions of hair preservation surgical device 100.

Referencing FIG. 6, in the illustrated embodiment, the anchor assembly 148 can include a through-hole or opening 150 that extends through the top and bottom walls 102, 104 of the hair preservation surgical device 100. The through-hole or opening 150 can have a variety of cross-sectional shapes and sizes. For example, according to illustrating embodiment, the through-hole or opening 150 has a generally rectangular or square cross-sectional shape. In such an embodiment, the through-hole or opening 150 can provide a location at which one or more anchors can extend from the body portion 114, as well as provide areas for locations at which one or more attachment mechanisms can extend around at least a portion of an anchor 152, 154 and through through-hole or opening 150.

More specifically, in the exemplary embodiment shown in at least FIG. 6, the through-hole or opening 150 of each anchor assembly 148 can provide a location for positioning of at least one first anchor 152 and, optionally at least one second anchor 154. For example, as seen in at least FIG. 6, the at least one first anchor 152 comprises a first pair of anchors 152, and the at least one second anchor 154 comprises a second pair of anchors 154. Further, the first anchors 152 may, or may not, have a shape, size, configuration, and/or orientation that is different than that of the second anchors 154. For example, as seen by at least FIG. 6, the first pair of anchors 152 can inwardly extend into the through-hole or opening 150 in a direction (d₂) that is generally parallel to the central longitudinal axis 122, while the second pair of anchors 154 can inwardly extend into the through-hole or opening 150 in a direction (d₁) that is generally parallel to the midline 140 of the body portion 114. The distance to which each first anchor 152 extends into the through-hole or opening 150 may come or may not be similar to at least one other anchor 154.

According to the exemplary embodiment shown in at least FIG. 6, the first pair of anchors 152 each have a width across the anchor 152 that is larger than a corresponding with across each anchor 154 the second pair of anchors 154. Such differences in size, such as, for example, width across a top portion 158 of the anchors 152, 154, can be configured to accommodate engagement by different types of, or different sized, attachment mechanisms. For example, referencing FIG. 8, each of the first pair of anchors 152 can be sized for use with an attachment mechanism 156 in the form of a staple having a certain crown size that extends between opposing legs of the staple and over a top portion 158 of the anchor 152. Similarly, the second pair of anchors 154, including the top portion 158 of the anchor 154, can be sized for staples having a crown size that is smaller than the crown size of the staples used with the first pair of anchors 152. Alternatively, while one of the first anchors 152 and the second anchors 154 can be sized for engagement with a staple, the other of the first anchors 152 and the second anchors 154 can be sized for use with a different type of attachment mechanism(s), including, but not limited to, a suture.

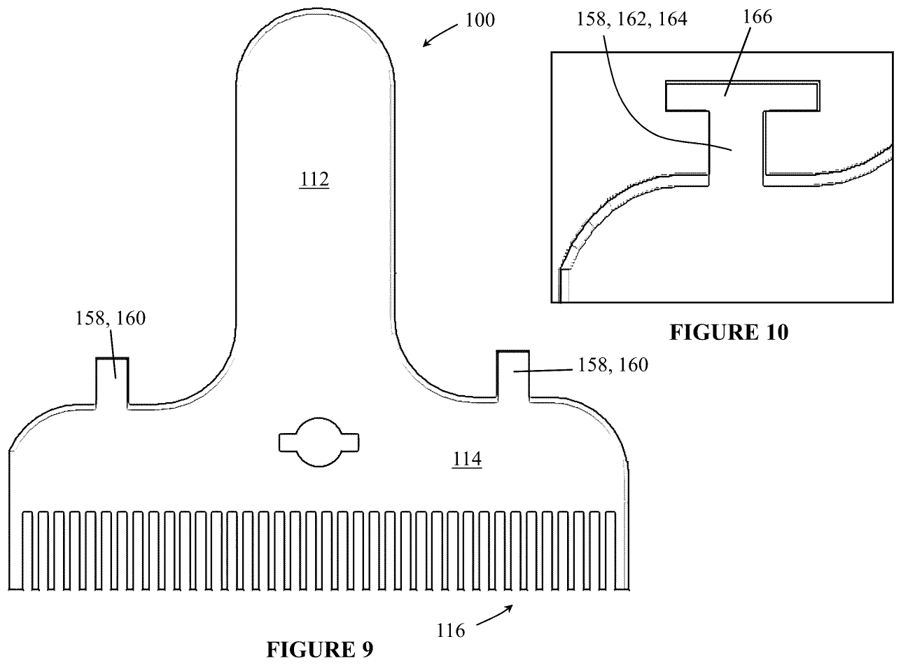

The anchors 152, 154 can be configured for a variety of different types of engagements with associated attachment mechanisms 156. For example, according to certain embodiments, the anchors 152, 154 can include what are more recesses, openings, or gaps that can accommodate passage of at least a portion of an attachment mechanism 156 through, as well as around, different portions of the anchor 152, 154. Additionally, rather than extending into a through-hole or opening 150, the anchors 152, 154 can outwardly extend from various positions about the body portion 114 or hair preservation surgical device 100. For example, FIG. 9 illustrates an embodiment in which a pair of anchors 160 outwardly project from a portion of the sidewall 106 around the first end 130 of the body portion 114 in a direction that is generally parallel to, and are on opposing sides of, the central longitudinal axis 122. However, such anchors 160 can be positioned at a variety of other locations, including at different portions of the body portion 114 of the hair preservation surgical device 100, and at directions that can be non-parallel, including perpendicular, to the central longitudinal axis 122. For example, according to certain embodiments, the anchors 160 can outwardly extend from the body portion 114 of the hair preservation surgical device 100 in a direction that is generally parallel to the midline 140 of the body portion 114, among other angular orientations.

While FIGS. 6 and 9 generally illustrate the anchors 152, 154, 160 as having a square or rectangular cross-sectional shapes, the anchors 152, 154, 160 can have a variety of other shapes and configurations. For example, FIG. 10 illustrates an alternative embodiment of FIG. 9 in which the anchor 162 has a generally T-shape. According to such an embodiment, the anchor 162 can have a lower leg portion 164 that extends in a direction that is generally parallel to the central longitudinal axis 122, and an upper cross leg 166 that extends in a direction that is generally perpendicular to the central longitudinal axis 122. Such an embodiment can, for example, assist in retaining engagement between the anchor 162 and the attachment mechanism 156. For example, the upper cross leg 166 and the first end 130 of the body portion 114 can be at opposing ends of the lower leg portion 164 and sized so as to provide barriers that can assist with preventing the lower leg portion 164 from sliding out from under an adjacent attachment mechanism 156 including, for example, from sliding from engagement with a staple or suture that may be positioned over the top portion 158 of the lower leg portion 164. However, the anchors 152, 154, 160, 162 can have a variety of other configurations that can at least assist in maintaining an engagement between the anchor 152, 154, 160, 162 and an associated attachment mechanism 156, including, for example, one or more open ended or closed ended apertures, such as, for example, generally C-shaped or O-shaped anchors, among other anchor configurations.

During use, a user holding the handle portion 112 of the hair preservation surgical device 100 can displace the hair preservation surgical device 100 relative to the head, or scalp, of the patient and with a force such that the hair engagement bodies 116 can engage and pull at least some of the hair strands of the patient. Further, the hair engagement bodies 116 can, via at least the force exerted upon the hair preservation surgical device 100 by the user pulling the hair strands, form a part between hair strands that are engaged with the hair preservation surgical device 100 and adjacent hair strands that are not engaged with the surgical device 100.

FIG. 8 illustrates an example of the hair preservation surgical device 100 secured to the scalp of a patient in a manner that can at least assist in pulling hair strands 170 away from other hair strands to form a part 168 therebetween in the patient's hair, as well as retain the pulled hair in a generally taut condition. As seen, the part 168 can provide an area of separation between adjacent portions of hair strands 170 such that at least skin covering the scalp 172 is viewable. At least a portion of the part 168 can be formed by the user, such as, for example, a surgeon, exerting a force against the hair strands 170 of the patient, such as, for example, a pulling force that may result in the deflection, deformation, or bending of the hair preservation surgical device 100 along the central longitudinal axis 122 of the hair preservation surgical device 100, as previously discussed and illustrated in FIG. 7.

Additionally, the hair preservation surgical device 100 can be displaced about, or relative to, the patient's head such that the hair preservation surgical device 100 is positioned to be secured to the patient at a location at which the access aperture 138 is positioned over, or generally aligns with, the part 168 that separates portions of the patient's hair strands. As previously discussed, such positioning of the access aperture 138 over the part 168 while the hair preservation surgical device 100 pulls at least some of the hair strands 170 away from the part can assist in preventing hair from interfering with a surgical procedure that will be performed through the access aperture 138.

With the hair strands 170 pulled in a direction that can least assist in providing, if not maintaining, the part 168 in the patient's hair, and the access aperture 138 positioned over the part 168, the user can subsequently secure the hair preservation surgical device 100 to the patient's scalp 172 via use of one or more attachment mechanisms 156. For instance, as shown by the example provided in FIG. 8, an attachment mechanism 156 in the form of a staple straddles the illustrated anchors 152, 154 and can enter into a least a portion of the patient's head.

The user can then proceed with performing one or more surgical procedures through the access aperture 138 of the secured hair preservation surgical device 100. For example, with the hair preservation surgical device 100 maintaining hair strands 170 generally away from the part 168, a cutting end of a cutting tool can extend through the access aperture 138 to cut an incision at an incision site, and a cranial drill bit can subsequent pass through the access aperture 138 to access, and pass through, the incision site. Other surgical procedures can also be performed through the access aperture 138, including, for example, placement of an electrode in a bore or hole drilled in at least the patient's skull. Similarly, with respect to surgical procedures that involve the placement of electrodes through a portion of a drilled bore in the skull of a patient, the hair preservation surgical device 100, and, more specifically, a replacement or other hair preservation surgical device 100, can also later be used for the removal of such electrodes. For example, in the context of such a removal procedure, a previously unused hair preservation surgical device 100 can be used to at least assist in again forming, and maintaining, a part 168 between adjacent portions of the patient's hair strands 170 and a location at which an electrode is placed. Further, such placement of the hair preservation surgical device 100, and associated securing of the hair preservation surgical advice to the scalp 172 of the patient, can involve positioning the hair preservation surgical device 100 at a location at which the access aperture 138 is generally positioned over the part 168 such that the access aperture 138 is generally aligned with the implanted electrode. With the access aperture 138 generally positioned over, and/or aligned with, the electrode, the user, including, for example, a surgeon, can proceed with removing the electrode from the patient through the access aperture 138.

While the disclosure has been illustrated and described in detail in the foregoing drawings and description, the same is to be considered as exemplary and not restrictive in character, it being understood that only illustrative embodiments thereof have been shown and described and that all changes and modifications that come within the spirit of the disclosure are desired to be protected.

1. An apparatus for forming a part along, and controlling a position of, at least a portion of a plurality of strands of hair of a patient, the apparatus comprising:
a handle portion configured to be grasped by at least one of a digit and a palm of a user;
a body portion coupled to the handle portion and comprising a plurality of hair retention bodies positioned and configured to engage and retain a position of the plurality of strands of hair;
an access aperture that extends through at least one of the body portion and the handle portion and is positioned to extend over the part; and
at least one anchor configured to be at least partially straddled by an attachment mechanism that secures the apparatus to a head of the patient.

2. The apparatus of claim 1, wherein the apparatus extends between a first end and a second end of the apparatus, and wherein the hair retention bodies inwardly extend from the second end of the apparatus toward the first end of the apparatus in a direction that is generally parallel to a central longitudinal axis of the apparatus.

3. The apparatus of claim 1, wherein the access aperture includes an orifice that extends through both a top wall and a bottom wall of the apparatus.

4. The apparatus of claim 3, wherein the access aperture further includes at least one slot that outwardly extends from the orifice and through both the top wall and the bottom wall.

5. The apparatus of claim 4, wherein the at least one slot comprises a first slot and a second slot positioned on opposing sides of the orifice.

6. The apparatus of claim 5, wherein the first slot, the second slot, and the orifice are centrally positioned along a midline of the body portion.

7. The apparatus of claim 1, wherein the at least one anchor inwardly extends into a through-hole in the apparatus.

8. The apparatus of claim 7, wherein the through-hole is positioned in the body portion.

9. The apparatus of claim 8, wherein the at least one anchor comprises a pair of anchors.

10. The apparatus of claim 1, wherein the at least one anchor has a square, rectangular, or non-circular cross sectional shape.

11. The apparatus of claim 1, wherein the at least one anchor is positioned between the hair retention bodies and a first end of the apparatus.

12. The apparatus of claim 1, further including at least one anchor assembly comprising a plurality of anchors that inwardly extend through an orifice in the body portion.

13. The apparatus of claim 12, wherein the at least one anchor assembly comprises a first anchor assembly and a second anchor assembly, and wherein the access aperture is positioned between the first and second anchor assemblies.

14. The apparatus of claim 1, wherein the apparatus has a thickness that accommodates bending, deflection, or deformation of the apparatus.

15. The apparatus of claim 1, further including a transition region between the handle portion and the body portion.

16. A method comprising:
engaging a plurality of hair strands of a patient with at least one of a plurality of hair engagement bodies of a hair preservation surgical device;
pulling the plurality of engaged hair strands via displacement of the hair preservation surgical device to form a part between the plurality of engaged hair strands and other hair strands of the patient;
positioning an access aperture of the hair preservation surgical device above the part; and
engaging one or more attachment mechanisms with one or more anchors of the hair preservation surgical device to securely couple the hair preservation surgical device to the patient.

17. The method of claim 16, further comprising inserting, while the hair preservation surgical device is securely coupled to the patient, a cutting edge of a cutting tool through the access aperture.

18. The method of claim 16, further including inserting, while the hair preservation surgical device is securely coupled to the patient, a cranial drill bit through the access aperture.

19. The method of claim 16, wherein pulling the plurality of engaged hair strands via displacement of the hair preservation surgical device further comprises deforming the hair preservation surgical device along a central longitudinal axis of the hair preservation surgical device to provide a force to pull the hair strands of the patient away from other hair strands.

20. The method of claim 16, wherein the attachment mechanisms comprises at least one of a staple and a suture.
